(19) United States
(12) Patent Application Publication
(10) Pub. No.: US 2004/0049206 A1
(43) Pub. Date: Mar. 11, 2004

(54) Scalp tensioner

(76) Inventor: William R. Rassman, Los Angeles, CA (US)

Correspondence Address: FITZPATRICK CELLA HARPER & SCINTO, 30 ROCKEFELLER PLAZA, NEW YORK, NY, 10112, US

(21) Appl. No.: 10/235,563

(22) Filed: Sep. 6, 2002

(51) Int. Cl. A61B 17/50

(52) U.S. Cl. 606/133

(57) ABSTRACT

A scalp tensioner for tensioning a scalp in a donor region of hair grafts, so as to facilitate harvesting of hair grafts. The tensioner includes at least two generally opposed support structures arranged to form an open space therebetween, and plural gripping mechanisms such as a linear array of teeth or needles so as to grip the scalp, with one such gripping mechanism being provided on each support structure. The support structures are slidingly engaged on guide rods, and coil springs coaxially mounted on the guide rods bias the support structures away from each other. The support structures are movable against the biasing action of the coil springs between a closed position at which the gripping mechanisms are engaged with the scalp, and an opened position which corresponds to a scalp tensioning position, and in which hair grafts can be harvested from the open space between the support structures.

### BACKGROUND OF THE INVENTION

1. Field of the Invention

The present invention relates to harvesting of hair grafts in preparation for hair transplantation, and in particular relates to a scalp tensioner which tensions the scalp at the harvest site so as to facilitate harvesting of the hair grafts.

2. Description of the Related Art

With increasingly better surgical techniques and refined procedures, hair transplantation has become an increasingly popular treatment for male pattern baldness. According to this technique, hair grafts from a donor site are usually taken in a fully-haired region at the lower scalp of the patient and then are harvested and the hair grafts are transplanted to a recipient site.

Although many harvesting techniques have been used, one older, popular technique uses a simple punch-harvest of the hair graft. According to this technique, the donor site is prepared by close cutting of the hair, leaving only a hair stub which serves as a marker to the surgeon so as to locate potential grafts. Then, a generally circular punch (rotating or non-rotating) is positioned over a desired harvest site, as shown in FIG. 1A, which in cross-section shows punch 10 positioned over harvest site 111 in scalp 12 over skull 14. The surgeon then presses punch 10 through scalp 12 until the proper depth is obtained, whereafter the punch and the harvested hair graft are withdrawn.

This punch-harvesting technique was initially developed at a time when hair grafts were formed of plugs of a large number of hairs, such as 20 to 30 hairs each. More recent hair transplantation techniques have achieved more realistic and natural results through the use of very small grafts, where each plug contains only a few hairs. To accomplish this, if the diameter of the punch has decreased, it leads to difficulties in the punch-harvest technique.

FIG. 1B illustrates one such difficulty. As seen there, as the punch 10 is depressed onto the scalp, scalp 12 deforms causing the target graft 15 to deflect in a more-or-less unpredictable way. Then, as the surgeon continues to depress punch 10, the punch 10 often will amputate the lower end 15a of the graft as illustrated by dotted line 16. Such a graft may be non-viable and may fail after transplantation.

This result is well-understood by skilled hair transplantation surgeons. To harden the scalp, such surgeons generally utilize tumescence, rock-hard tumescence or apply lateral hand pressure to the scalp. Tumescence is a technique whereby fluid is injected directly into tissue, skin, and the area below the skin, to such a degree that the injection area swells as a result of the volume of fluid injected. The amount of swelling is directly proportional to the amount of fluid injected, however, within seconds of the injection, much of the fluid diffuses out of the local area. Rock-hard tumescence occurs when an area is rapidly injected with a large volume of fluid. These skilled surgeons understand that a hardened scalp provides less latitude for the scalp to deform during punch-harvesting, with consequent reduction in the deformation of the skin when the grafts are taken. However, because tumescence, as well as rock-hard tumescence, is temporary, lasting only seconds, good hair graft harvesting is highly dependent on a small window of time. Therefore, recurring tumescence is required, making this approach unacceptable for any significant number of grafts.

### SUMMARY OF THE INVENTION

It is an object of the invention to provide a scalp tensioner which provides automatic tensioning of the scalp in the area of a donor site so as to facilitate harvesting of hair grafts, preferably with a higher proportion of viable grafts as compared to tumescence or manual tensioning.

According to one aspect, a scalp tensioner according to the invention includes two or more generally opposed support structures which are arranged to form an open space therebetween, with each support structure having a gripping mechanism for gripping the scalp. A biasing mechanism biases the support structures outwardly away from each other, yet also permits movement of the support structures toward each other for placement on the scalp.

In use, the support structures and their corresponding gripping mechanisms are moved toward each other against bias by the biasing mechanism. The gripping mechanisms are engaged with the scalp, and the biasing mechanism is activated so as to tension the scalp, whereby the open space between the support structures permits harvesting of hair grafts from the tensioned scalp.

Preferably, the support structures are slidingly engaged on guide rods, and the biasing mechanism is comprised of a coil spring coaxially mounted around the rods. Such a structure permits the surgeon to grip the support structures and move them toward each other against the coil spring, and thereafter engage the gripping mechanism with the scalp and permit the coil springs to expand and tension the scalp.

Good results are also obtainable when the biasing mechanism is a geared, ratchet, screw or head brace mechanism.

In particularly preferred embodiments, the gripping mechanism is a linear or planar array of needles, teeth, or other pointed structures that pierce the epidermis of the scalp so as to grip it. Effective results are also obtained when the gripping structure is hair clamp which clamps uncut hair in the vicinity of the donor site.

This brief summary has been provided so that the nature of the invention may be understood quickly. A more complete understanding of the invention can be obtained by reference to the following detailed description of the preferred embodiment thereof in connection with the attached drawings.

### BRIEF DESCRIPTION OF THE DRAWINGS

FIGS. 1A and 1B are views showing a conventional punch-harvesting procedure for hair grafts.

FIG. 2 is a perspective view showing a scalp tensioner according to a preferred embodiment of the invention.

FIG. 3 is a cross-sectional view illustrating harvesting of hair grafts using a scalp tensioner according to the invention.

FIG. 4 is a view showing a second embodiment of a scalp tensioner according to the invention.

FIGS. 5 and 6 are cross-sectional and perspective views, respectively, showing a third embodiment of a scalp tensioner according to the invention.

FIG. 7 is a perspective view showing a fourth embodiment of a scalp tensioner according to the invention.

FIGS. 8, 9 and 10 are cross-sectional views illustrating using a scalp tensioner according to the fourth embodiment of the invention.

FIGS. 11 and 12 are perspective views showing a fifth embodiment of a scalp tensioner according to the invention.

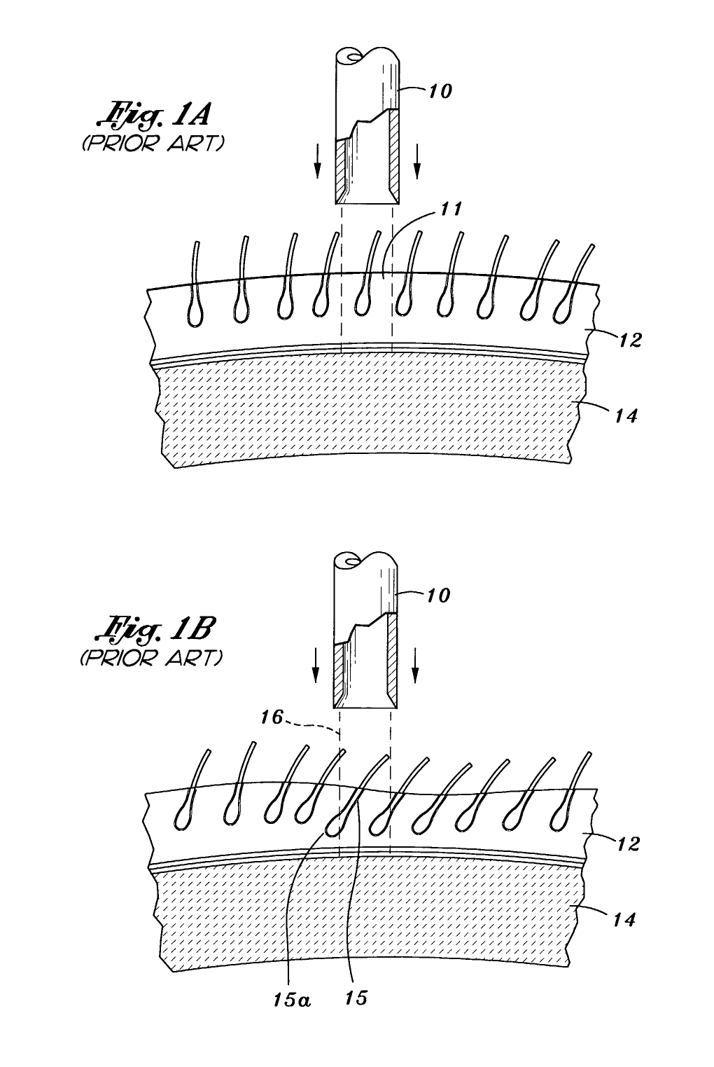

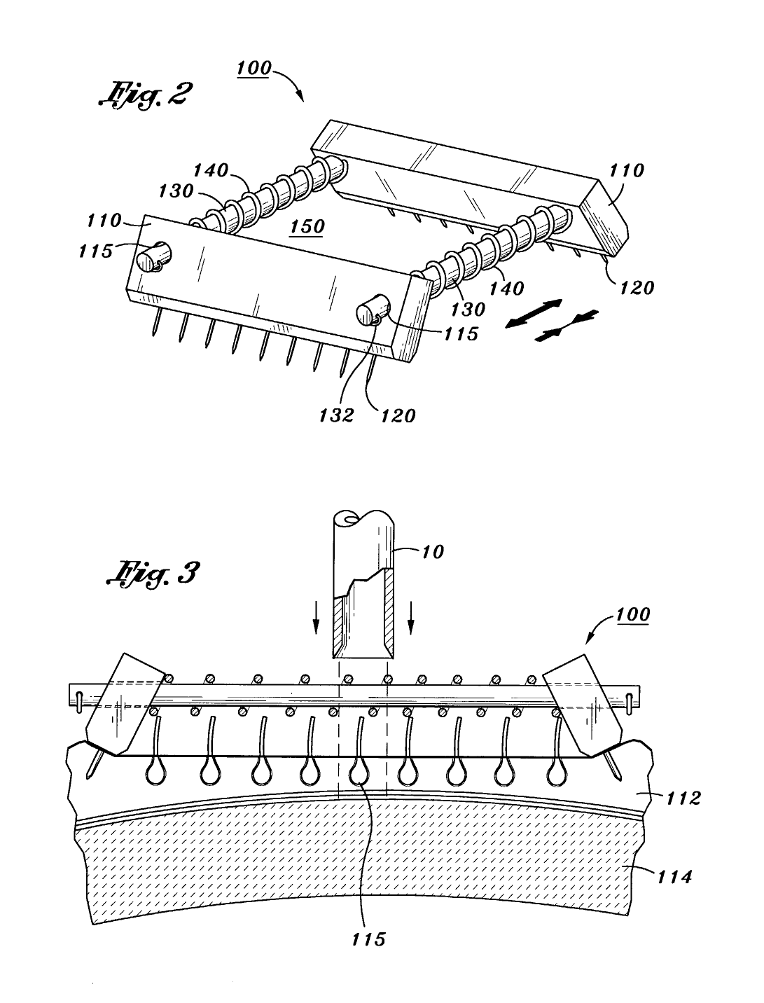

### DETAILED DESCRIPTION OF THE PREFERRED EMBODIMENTS

FIG. 2 is a perspective view of a scalp tensioner according to one preferred embodiment of the invention. As shown in FIG. 2, scalp tensioner 100 includes a pair of support structures 110 generally opposed to each other and formed of a surgically sterilizable material which in this embodiment is light weight aluminum formed into blocks. Each support structure 110 supports a gripping mechanism 120 which in this embodiment is formed of a linear array of stainless steel needles or rods sharpened to a point. Through-holes 115 on support structures 110 slidingly receive guide rods 130, each having a stop 132 to ensure that the support structures 110 remain on the guide rods 130. In this embodiment, the stop is a cotter ring but other arrangements, such as a flared end, are also possible. The guide rods are formed from aluminum.

Coil springs 140 form a biasing mechanism for biasing the support structures 110 away from each other yet allowing movement between a closed position in which the support structures are generally close to each other, and an open position which corresponds to a scalp tensioning position. The coil springs 140 are formed of stainless steel and are coaxially mounted around guide rods 130.

FIG. 2 shows a scalp tensioner in its open position in which an open space, generally designated at 150, is formed between the support structures 110. In the open position, the open space permits harvesting of hair grafts from a scalp tensioned by the device.

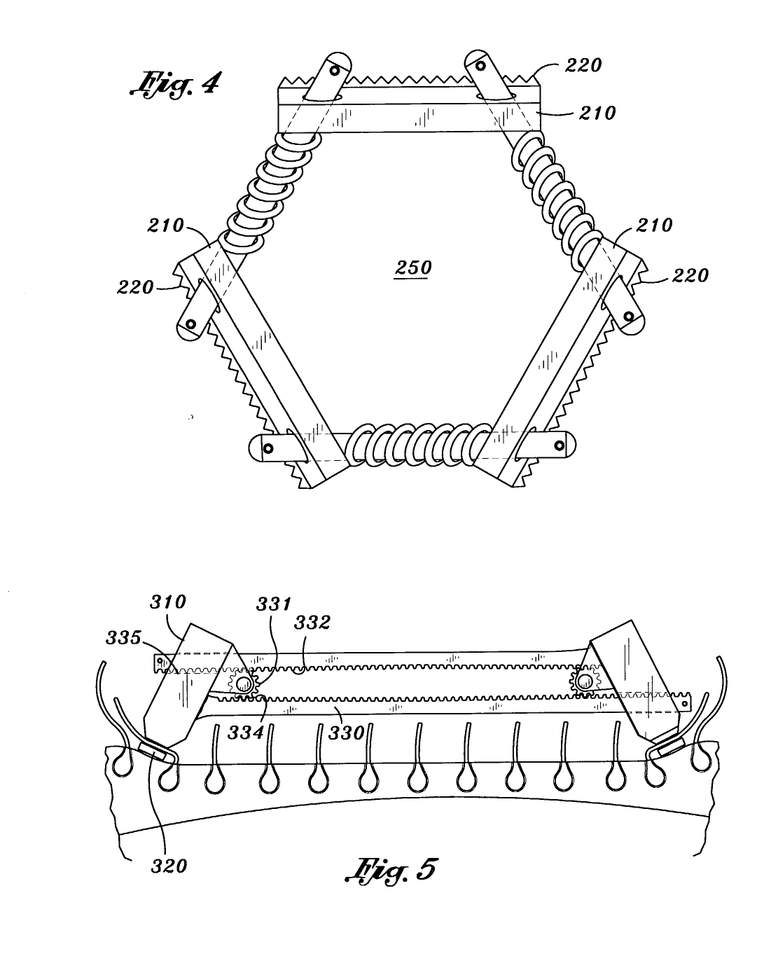

To use the scalp tensioner shown in FIG. 2, a surgeon grips support structures 110 and moves them to the closed position, against the biasing force of coil springs 140. Gripping mechanisms 120 are then engaged against the scalp so as to pierce it, and the surgeon's grip is released. Coil springs 140 thereafter bias the tensioner to its open position, as shown generally in FIG. 3. As seen there, the scalp in the donor region is tensioned laterally outwardly, which causes a thinning of scalp 112 against skull 114, and a general straightening and alignment of hair grafts 115. Thereafter, a punch-harvester 10 is aligned over a hair graft, and pushed in a downward direction. Because of the tensioning effected by scalp tensioner 100, the scalp 112 and the target hair graft 115 do not deform significantly, thereby allowing a clean punch-harvesting of the target graft.

FIG. 4 shows an alternative embodiment of the present invention. As seen in FIG. 4, this embodiment features more than two support structures, in this case three support structures 210. The support structures are generally opposed, and in this case form a generally triangular open region 250. Because the FIG. 4 embodiment tensions in three directions rather than the single direction of the FIG. 2 embodiment, superior tensioning may be achieved.

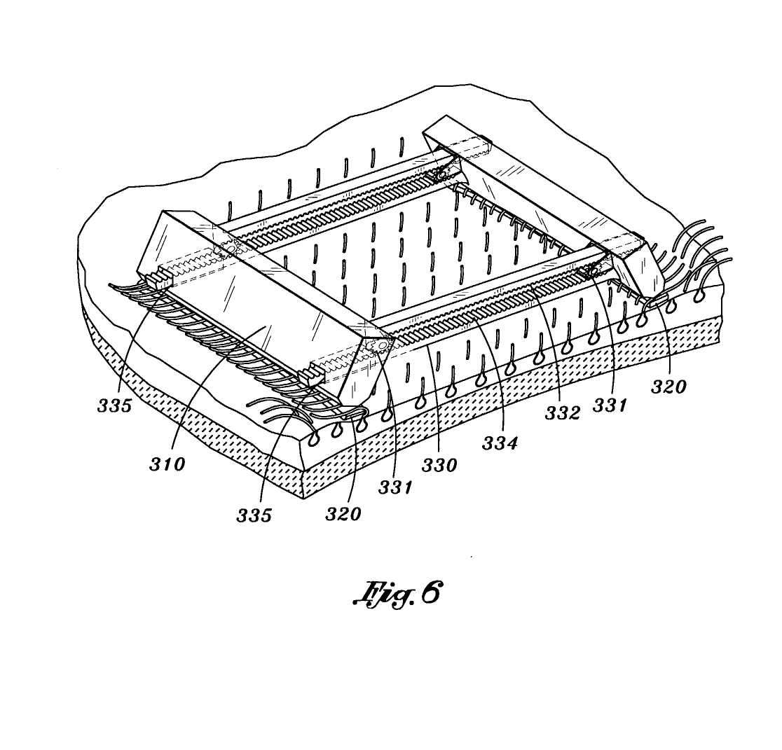

The FIG. 4 embodiment also uses a linear array of teeth as a gripping mechanism. Other gripping mechanisms are also usable, as described below in connection with the embodiment shown in FIGS. 5 and 6.

Various arrangements are also possible in which the gripping mechanism on one support structure differs from the gripping mechanism on another support structure, such as teeth on one support structure and needles on another. It is also possible to arrange one of the support structures to be fixed with respect to its guide rods, with the remaining supporting structures being movable on the guide rods relative to the fixed support structure.

FIGS. 5 and 6 illustrate a further embodiment of the invention in which the biasing mechanism is formed from a geared assembly 330 in which a central gear 331 engages with opposed gear faces 332 and 334 on a pair of opposed guide rods 335. Using this geared ratchet assembly, support structures 310 are able to move between a closed position and an open position, and be biased in the open position by the geared ratchet assembly 330.

This embodiment also features a further example of a gripping mechanism, in the form of a hair clamp. In particular, hair clamp 320 clamps uncut hair at the periphery of the donor region of otherwise closely-cut hair, so as to allow the scalp tensioner to adhere to the scalp.

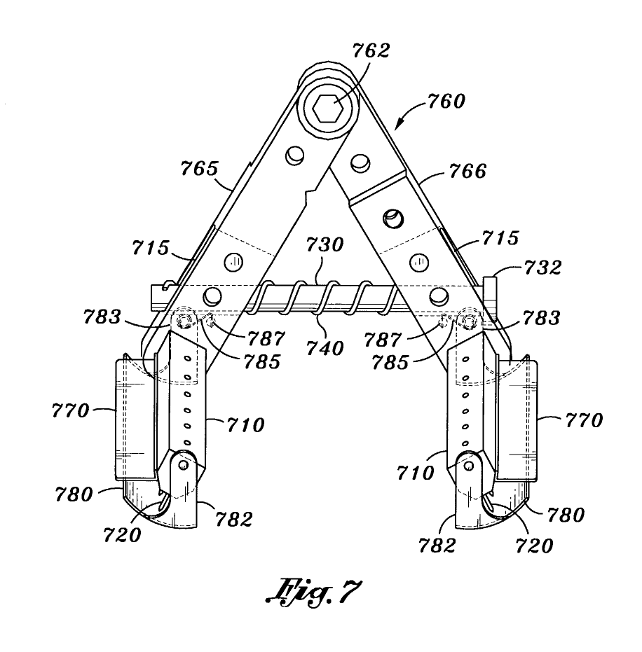

FIG. 7 demonstrates another embodiment of the present invention. As seen in FIG. 7, this embodiment features a biasing mechanism incorporating hinge assembly 760. Pivoting hinge arms 765 and 766 of hinge assembly 760 attach at hinge pivot 762. Through-holes 715 in pivoting hinge arms 765 and 766 house opposite ends of coil spring 740 which is coaxially mounted around guide rod 730. Guide rod 730 has stop 132 to ensure that hinge arm 766 remains on the guide rod 730.

Support structures 710 are secured perpendicularly to the unhinged free ends of pivoting hinge arms 765 and 766 and are extended in the same direction to be generally opposed to each other. Stationary gripping mechanism guards 770 extend from support structures 710 in a direction opposite the opposing support structure 710 to form a safety partition guarding gripping mechanisms 720. Moveable gripping mechanism guards 780 are also pivotally attached to support structures 710 to form another safety partition guarding gripping mechanisms 720. A first moveable gripping mechanism guard arm 782 is pivotally attached the free end of support structures 710. A second moveable gripping mechanism guard arm 783 is pivotally attached to pivoting hinge arms 765 and 766 opposite the support structures 710. Moveable gripping mechanism guards 780 are biased to a default position adjacent stationary gripping mechanism guards 770 through attachment to biasing springs 785. Biasing springs 785 are further secured to biasing spring anchors 787. Biasing spring anchors 787 are attached to pivoting hinge arms 765 and 766 generally opposite the support structures 710.

The surface of moveable gripping mechanism guards 780 opposite gripping mechanisms 720 is textured or serrated so as to grip the scalp in a manner that disfavors lateral slipping when biased toward and along the surface of the scalp. Various constructions are also possible in which the moveable gripping mechanism guards 780 are solid, mesh or grill.

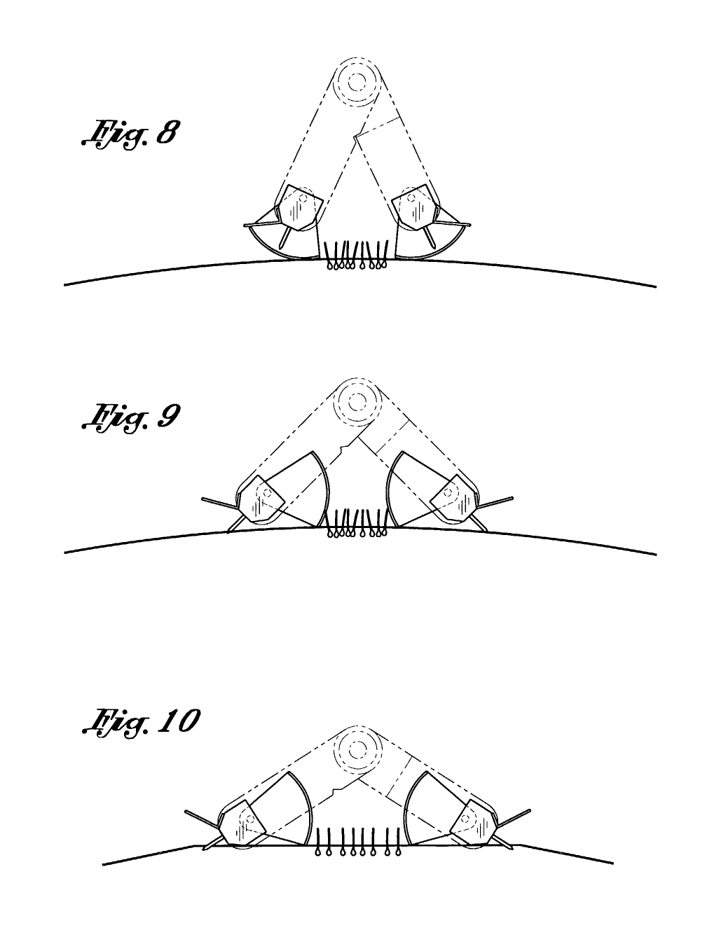

To use the embodiment of the scalp tensioner shown in FIG. 7, a surgeon grips support structures 710 and moves them to the closed position, against the biasing force of coil spring 740. Moveable gripping mechanism guards 780 are then engaged against the scalp and pressure is applied toward the scalp using the tensioner, as generally shown in FIG. 8. The surgeon's grip biasing the closed position is then released while maintaining the pressure toward the scalp with the tensioner. The coil spring 740 thereafter biases the tensioner toward its open position. The textured surface of the moveable gripping mechanism guards 780 grip the scalp and pivot inward toward one another as the tensioner opens. Gripping mechanisms 720 are exposed and move away from each other along the scalp and lower until contact with the scalp is achieved, as generally shown in FIG. 9. Thus, constant pressure toward the scalp from the tensioner as the coil spring 740 biases the tensioner toward its open position results in the recession of moveable gripping mechanism guards 780 and gripping mechanisms 720 engaging the scalp, as generally shown in FIG. 10 which demonstrates the utilization of a linear array of needles as gripping mechanisms 720.

Once the surgeon disengages the scalp tensioner from the scalp, the moveable gripping mechanism guards 780 automatically return to their default position baised against stationary gripping mechanism guards 770.

This embodiment therefore safely covers the gripping mechanisms 720 when not in use as well as allows the surgeon to access the donor region of the scalp from an area perpendicular to the donor region as well as an area lateral to the donor region. This access permits harvesting of hair grafts from a scalp tensioned by the device from multiple directions.

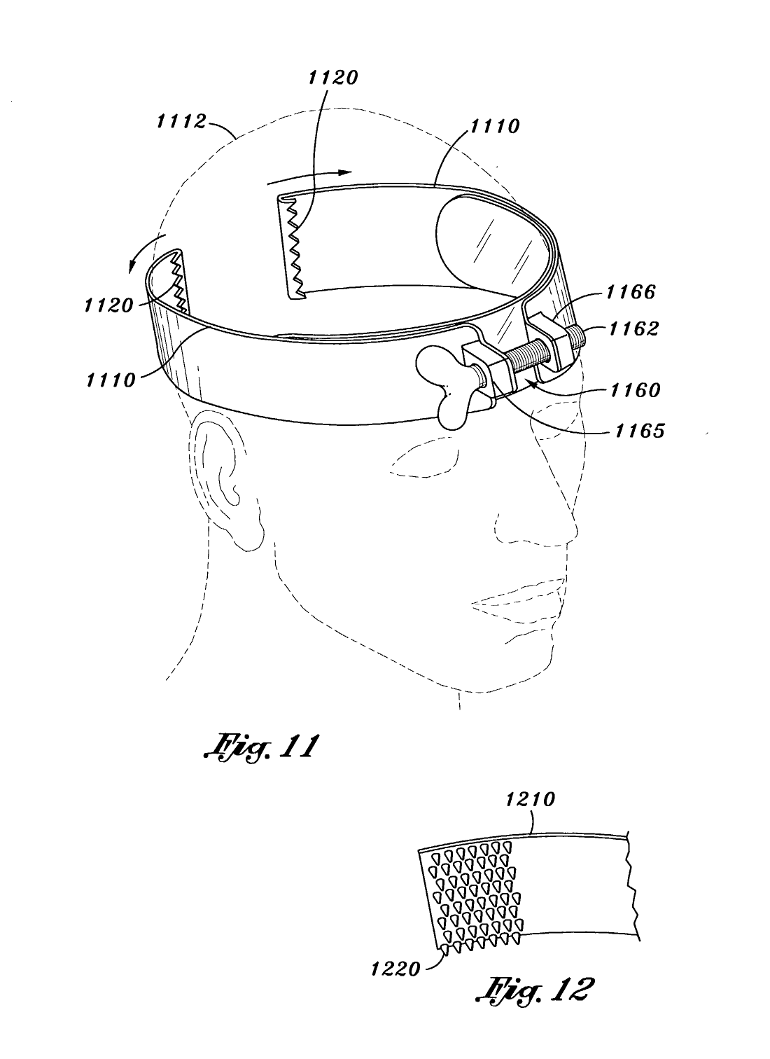

FIG. 11 shows a further embodiment of the invention. As illustrated in FIG. 11, this embodiment features support structures 1110 formed from a band like head brace. Support structures 1110 are integrated with a biasing mechanism incorporating screw assembly 1160. Screw 1162 is engaged with support structures 1110 through nuts 1165 and 1166. Gripping mechanisms 1120 are placed at free ends of support structures 1110.

To use the embodiment of the scalp tensioner shown in FIG. 11, a surgeon unscrews screw 1162, loosening support structures 1110 to the closed position. The scalp tensioner is then placed circumferentially around the perimeter of head and oriented so that the donor region is accessible between gripping mechanisms 1120. The surgeon engages gripping mechanisms 1120 with the scalp and screws screw 1162 through nuts 1165 and 1066 contracting support structures 1110 toward each other at screw 1162 and biasing the tensioner toward its open position resulting in tensioning scalp 1112.

Various arrangements are also possible in which three or more band like support structures encompass various regions of the head providing for numerous gripping mechanism engagement areas.

FIG. 12 illustrates another possible arrangement of this embodiment in which the gripping mechanism 1220 on support structure 1210 is formed from a planar array of needles.

The invention has been described with respect to particular illustrative embodiments. It is to be understood that the invention is not limited to the above-described embodiments and that various changes and modifications may be made by those of ordinary skill in the art without departing from the spirit and scope of the invention. The various embodiments can be combined with each other to yield embodiments different from those shown herein. As one example, a feature or features from one embodiment can be substituted for corresponding features in another embodiment. In particular, gripping mechanisms can be substituted, and they can be substituted asymmetrically such that a gripping mechanism on one support structure differ from that on another support structure. Thus, the invention should not be limited to the disclosed structures but rather should be measured by the appended claims.

What is claimed is:

1. A scalp tensioner comprising:
at least two support structures generally opposed to each other and arranged to form an open space therebetween;
plural gripping mechanisms, one such gripping mechanism on each of said support structures, and each said gripping mechanism constructed to grip the scalp; and
a biasing mechanism for biasing said support structures away from each other, and permitting movement of said support structures between a closed position and an opened position which corresponds to a scalp tensioning position;
wherein in the opened position, said open space permits harvesting of hair grafts from a tensioned scalp.

2. A scalp tensioner according to claim 1, wherein there are exactly two support structures.

3. A scalp tensioner according to claim 1, wherein there are exactly three support structures.

4. A scalp tensioner according to claim 1, wherein there are more than three support structures.

5. A scalp tensioner according to claim 1, further comprising guide rods on which said support structures are engaged.

6. A scalp tensioner according to claim 5, wherein said support structures are slidingly engaged on said guide rods, and wherein said biasing mechanism comprises coil springs coaxially mounted on said guide rods.

7. A scalp tensioner according to claim 1, wherein said support structures comprise straps of a band like head brace.

8. A scalp tensioner according to claim 1, wherein said biasing mechanism comprises a coil spring.

9. A scalp tensioner according to claim 1, wherein said biasing mechanism comprises a geared ratchet mechanism.

10. A scalp tensioner according to claim 1, wherein said biasing mechanism comprises a hinge assembly.

11. A scalp tensioner according to claim 1, wherein said biasing mechanism comprises a screw assembly.

12. A scalp tensioner according to claim 1, wherein said gripping mechanism comprises a linear array of needles.

13. A scalp tensioner according to claim 1, wherein said gripping mechanism comprises a linear array of teeth.

14. A scalp tensioner according to claim 1, wherein said gripping mechanism comprises a planar array of teeth.

15. A scalp tensioner according to claim 1, wherein said gripping mechanism comprises a planar array of needles.

16. A scalp tensioner according to claim 1, wherein said gripping mechanism is comprised of a hair clamp.

17. A scalp tensioner according to claim 1, further comprising stationary gripping mechanism guards.

18. A scalp tensioner according to claim 1, further comprising moveable gripping mechanism guards.

19. A scalp tensioner according to claim 18, wherein said moveable gripping mechanism guards comprise a textured surface.

20. A scalp tensioner according to claim 19, wherein said textured surface comprises a serrated surface.

21. A scalp tensioner according to claim 1, formed of surgically sterilizable materials.

22. A method for harvesting hair grafts using a scalp tensioner which includes at least two support structures generally opposed to each other and arranged to form an open space therebetween, plural gripping mechanisms with one such gripping mechanism on each of said support structures, and a biasing mechanism for outwardly biasing the support structures and for permitting movement of the support structures between a closed position and an opened position, said method comprising the steps of:
moving the support structures to the closed position;
engaging the gripping mechanisms with the scalp;
activating the biasing mechanism so as to bias the support structures to the opened position; and
harvesting hair grafts from the open space between the support structures.

23. A method according to claim 22, wherein said biasing mechanism comprises springs, and wherein said moving step includes the step of gripping the support structures and manually moving them to the closed position.

24. A method according to claim 23, wherein said activating step comprises the step of releasing the grip of the support structures.

25. A method according to claim 22, wherein said biasing mechanism comprises a geared ratchet assembly, and wherein said moving step comprises the step of engaging the gears to move the support structures to the closed position.

26. A method according to claim 25, wherein said activating step comprises the step of engaging the gears to move the support structures to the opened position.

27. A method according to claim 22, wherein said biasing mechanism comprises a screw assembly, and wherein said moving step comprises the step of loosening a screw.

28. A method according to claim 27, wherein said activating step comprises the step tightening a screw.

29. A method according to claim 22, wherein said activating step recesses moveable gripping mechanism guards.

30. A method according to claim 29, wherein said moveable gripping mechanism guards automatically return to a default position once said scalp tensioner is disengaged from the scalp.

31. A method according to claim 22, wherein said harvesting step comprises the step of punch-harvesting of hair grafts.

32. A method according to claim 31, wherein each said hair graft includes fewer than three to five hair follicles.
